(19) United States
(12) Patent Application Publication
(10) Pub. No.: US 2005/0245793 A1
(43) Pub. Date: Nov. 3, 2005

(54) Personal wellness monitor system and process

(76) Inventors: Theodore Craig Hilton, Columbus, NC (US); Jeff Willis, Spartanburg, SC (US)

Correspondence Address: DORITY & MANNING, P.A., POST OFFICE BOX 1449, GREENVILLE, SC, 29602-1449, US

(21) Appl. No.: 11/106,034

(22) Filed: Apr. 14, 2005

Related U.S. Application Data
(60) Provisional application No. 60/561,935, filed on Apr. 14, 2004.

(51) Int. Cl. A61B 5/00; A63B 71/00; A61B 5/02; A61B 5/103; A61B 5/117

(52) U.S. Cl. 600/300; 600/485; 600/500; 600/595; 482/8

(57) ABSTRACT

Disclosed is an apparatus and methodology for providing a personal wellness monitor (PWM) that may be used to measure and store physiological parameters indicative of sustained activity by a User. Data collected by the PWM may be correlated over time with relation to a particular activity mode. Activity modes monitored may include walking, sleeping, exercising, or other activities. The User may be alerted by the PWM as predetermined physiological parameters are reached. Data from the PWM may be uploaded to a Data Center and stored for analysis and historical record in a central database, accessible by the User. The User's historical record may be pertinent in determining changes in health and wellness.

### REFERENCE TO PREVIOUS APPLICATION

This application claims priority from previously filed Provisional Application No. 60/561,935 filed on Apr. 14, 2004.

### FIELD OF THE INVENTION

The present technology relates to apparatus and methods for monitoring the health of individuals. More particularly, the present technology provides apparatus and methods for providing continuous monitoring and evaluation of an individual's wellness.

### BACKGROUND OF THE INVENTION

The development of apparatus and methods for monitoring indications of an individual's health status is a continuously developing field that has been of interest for a long time. Early forms of health determination often required a trip to a health care provider or hospital where specialized equipment operated by trained personnel may be used to determine health related parameters for an individual. Such parameters might include relatively easily monitored conditions including, but not limited to, weight, blood pressure, pulse rate, and temperature. More sophisticated technology might be required for more advanced monitoring requirements such as blood oxygen level or blood and/or other body fluid composition determination.

Individuals who require prolonged health monitoring may be required to return to their health care providers on a regular basis or arrange for a nurse or other individual to visit them in order to be able to continuously monitor their health condition. Such home care can be expensive and inconvenient to all involved. Prior attempts have been made to address this home health care issue by developing home based monitoring equipment. Such equipment, however, is often expensive, bulky and difficult to operate. In some cases, home based equipment must be connected to a telephone or other communications connection and still may require the real time attention of a nurse or technician to instruct a home bound patient as to operational procedures.

Another area of concern in the area of health and wellness monitoring relates to long term monitoring. It may be important to be able to track an individual's ongoing health and wellness related parameters for extended periods of time in order to detect and evaluate developing trends. Such long-term activities may require repeat visits to or by a health care provider or long term use of home health monitoring equipment. In order to address the difficulties associated with such long-term monitoring, efforts have been made to develop portable monitoring devices. These efforts have not been entirely successful, however, as the devices developed have often been bulky and/or difficult to operate.

Yet another area of concern in the area of health and wellness monitoring relates to the more current interest in personal wellness monitoring and, in particular, to monitoring an otherwise healthy individual's wellness as such individuals seek to maintain or improve their physical well-being. Such individuals may simply wish to monitor physiological and biometric properties. Alternatively, they may be interested in embarking on various improvement regimens such as exercise programs where they may wish to track variations in physiological and biometric properties to assess the effectiveness of such programs. The use of known health care monitoring equipment or the protracted interaction with health care providers to implement monitoring activities for extended periods may not be practical or effective especially if real time monitoring is necessary or important.

While various implementations of health monitoring apparatus have been developed, and while various combinations of health and wellness information gathering methods have been developed, no design has emerged that generally encompasses all of the desired characteristics as hereafter presented in accordance with the subject technology.

### SUMMARY OF THE INVENTION

In view of the recognized features encountered in the prior art and addressed by the present subject matter, an improved apparatus and methodology for monitoring personal wellness on an ongoing or continuous basis has been developed.

In an exemplary configuration, a Personal Wellness Monitor (PWM) has been developed as a small electronic device that may be configured to be worn by an individual and to provide periodic measurements of selected physiological and biometric properties of the wearer. Measurements obtained may be stored in an onboard memory device for later retrieval. In one of its simpler forms, the PWM in accordance with the present technology may correspond to a wristband mounted device physically resembling a watch although other wear options are envisioned as will be discussed later.

One positive aspect of the PWM in accordance with the present technology is that a user may comfortably wear the device for extended periods. The ability to comfortably wear the device for extended periods provides not only easy of use but additional motivation to continue use of the PWM device to provide the long term data necessary to effectively monitor relatively slowly changing physiological and biometric properties as might occur from a regular and extended exercise program.

In accordance with aspects of certain embodiments of the present subject matter, methodologies are provided to selectively monitor different types of physical activities and to store individual sequential records based on each activity type. Activity types may be manually identified to set a mode of operation for the PWM device. In accordance with related aspects of the present technology, activity types may be automatically identified.

In accordance with certain aspects of other embodiments of the present subject matter, methodologies have been developed to provide visual indications based on real time analysis of data collected based on physiological and biometric properties of the PWM device user. Visual indications may be employed to assist the user in reaching and/or maintaining certain biometric targets.

In accordance with yet additional aspects of further embodiments of the present subject matter, apparatus and accompanying methodologies have been developed to collect and evaluate long term data to track the user's progress toward prescribed or target goals relating to his or her own personal wellness.

According to yet still other aspects of additional embodiments of the present subject matter, apparatus and methodologies have been developed to facilitate user data collection by providing convenient methodologies for transmitting collected data to a central processing facility for evaluation.

In accordance with yet still further aspects of still further embodiments of the present subject matter, methodologies have been developed to provide guidance to PWM users based on evaluations of collected data to assist the user in reaching his/her personal wellness goals.

Additional objects and advantages of the present subject matter are set forth herein or will be apparent to those of ordinary skill in the art from the detailed description herein. Also, it should be further appreciated that modifications and variations to the specifically illustrated, referred and discussed features and elements hereof may be practiced in various embodiments and uses of the invention without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

Still further, it is to be understood that different embodiments, as well as different presently preferred embodiments, of the present subject matter may include various combinations or configurations of presently disclosed features, steps, or elements, or their equivalents (including combinations of features, parts, or steps or configurations thereof not expressly shown in the figures or stated in the detailed description of such figures). Additional embodiments of the present subject matter, not necessarily expressed in the summarized section, may include and incorporate various combinations of aspects of features, components, or steps referenced in the summarized objects above, and/or other features, components, or steps as otherwise discussed in this application. Those of ordinary skill in the art will better appreciate the features and aspects of such embodiments, and others, upon review of the remainder of the specification.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a front view of an exemplary physical configuration of a Personal Wellness Monitor (PWM) in accordance with the present technology;

FIG. 1a illustrates a rear view of a Personal Wellness Monitor (PWM) in accordance with the present technology;

FIG. 2 illustrates the exemplary mounting arrangement of the Personal Wellness Monitor (PWM) of FIG. 1 on a wristband;

FIG. 3 schematically illustrates operational components of a exemplary Personal Wellness Monitor in accordance with the present technology;

FIG. 4 illustrates an exemplary configuration of a kiosk usable for data collection from a Personal Wellness Monitor; and

FIG. 5 diagrammatically illustrates an operational overview and system flow diagram of the general operation of the data gathering and evaluation aspects of the present technology.

Repeat use of reference characters throughout the present specification and appended drawings is intended to represent same or analogous features or elements of the invention.

### DETAILED DESCRIPTION OF THE PREFERRED EMBODIMENTS

As discussed in the Summary of the Invention section, the present subject matter is particularly concerned with an improved apparatus and methodology for monitoring personal wellness on an ongoing or continuous basis.

Selected combinations of aspects of the disclosed technology correspond to a plurality of different embodiments of the present invention. It should be noted that each of the exemplary embodiments presented and discussed herein should not insinuate limitations of the present subject matter. Features or steps illustrated or described as part of one embodiment may be used in combination with aspects of another embodiment to yield yet further embodiments. Additionally, certain features may be interchanged with similar devices or features not expressly mentioned which perform the same or similar function.

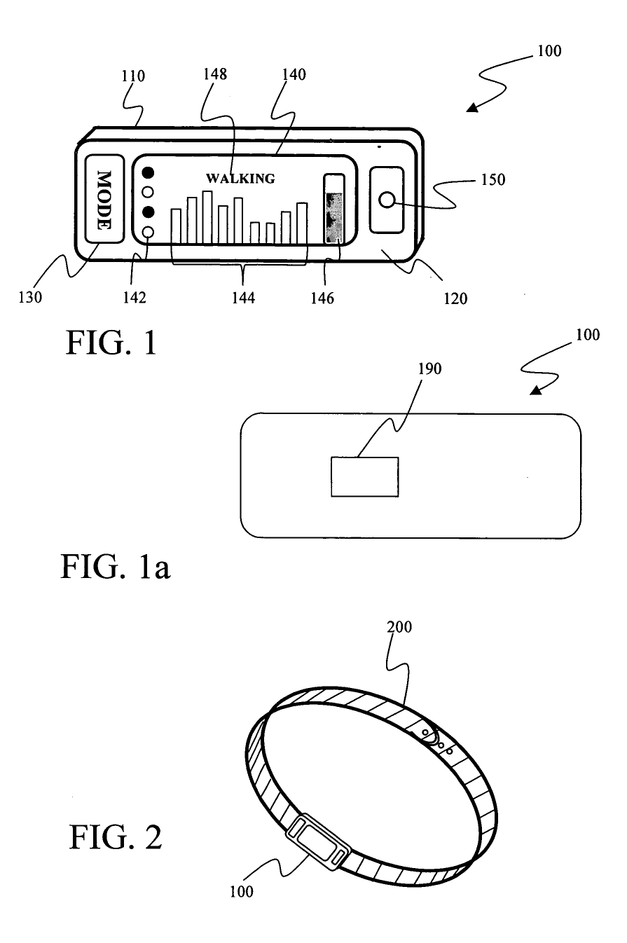

Reference will now be made in detail to the presently preferred embodiments of the subject Personal Wellness Monitor system and process. Referring now to the drawings, FIG. 1 illustrates an exemplary physical configuration of a Personal Wellness Monitor (PWM) 100 in accordance with the present technology. As may be seen from FIG. 1, the PWM 100 may be configured as a small electronic device that may be attached to the arm, wrist or other appendage of an individual user. In an exemplary embodiment, PWM 100 may be attached to an adjustable wristband 200 as illustrated in FIG. 2, however such user attachment method is not a specific limitation of the present technology it only being required that the PWM 100 be associated with the user in a manner permitting detection of particular selected parameters to be detected. In other exemplary embodiments of the present technology, a PWD 100 may be attached to the arm, wrist or other appendage of an individual user by way an expandable bracelet or cuff or any other suitable means.

In accordance with aspects of certain embodiments of the present technology a PWD 100 may have more than one assigned or registered user. The device may be separately correlated to each user by way of, for example, an incorporated radio frequency identification (RFID) chip, as will be discussed more fully later. Such correlation of device to user is applicable to all situations whether a PWM device is registered to single or multiple users. For ease of understanding, however, the present discussion will assume that a single user is registered to a single PWD 100.

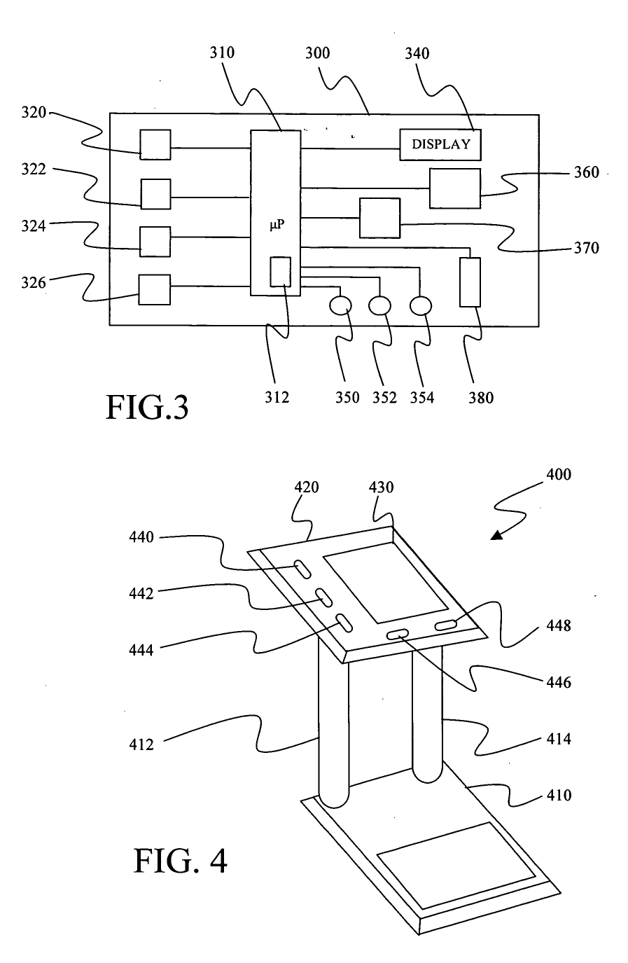

With further reference to FIG. 1, it will be seen that PWM 100 is provided with a housing 110 enclosing a number of electronic components as will be more fully described later with reference to FIG. 3. As illustrated in FIG. 1, PWM 100 is provided with a front panel 120 on which is mounted a number of control and display elements to provide a user with operational control of the PWM 100 as well as visual indications of data and other information as may be useful to the user. Principal components mounted on the front panel 120 to the PWM 100 include a mode switch 130, a display panel 140 and a visual indicator 150, each of which will be more fully described later.

With reference to FIG. 1a, a rear view of PWM 100 is shown and illustrates an exemplary location for a sensor array 190. Sensor array 190 may correspond to a number of individual sensors as will be more fully explained later with reference to FIG. 3, however, in brief, sensor array 190 may correspond to a number of individual sensors that provide, in cooperation with additional control circuitry, for the automatic and periodic measurements of physiological and biometric properties including heart pulse rate, blood pressure (systolic and diastolic), changes in motion (accelerometer), and other such physiological and biometric measurements as may be deemed important.

With further reference to FIG. 1, display panel 140 is configured to provide visual indications of the various physiological and biometric properties monitored by PWM 100 as well as PWM 100's operational status. Thus in an exemplary embodiment, display panel 140 may correspond to a liquid crystal display (LCD) and light emitting diode (LED) display combination or other suitable display device and may display information in the form of operational indicia 142, bar graph or other graphical information 144, battery charge level 146, and operational mode 148, in the illustrated example, a “walking” mode. These displays may optionally be provided as color displays where variations in color may be associated with different visual cues for the user.

The PWM 100 incorporates a paddle switch or other user-selectable mode switch 130 that allows a user to select among various activity modes. These modes may include (but are not limited to): sleep, eating, meditating, walking, jogging, swimming, weight training, yoga/pilates, profession (work), entertainment, driving, yard work, set resting pulse rate (5 minutes after arising from sleep), Wellness Score, relaxation (as defined by the user), auto select, and other such modes as may be deemed necessary or desirable.

With reference now to FIG. 3, there is illustrated an exemplary configuration of operational components 300 of an exemplary Personal WelIness Monitor (PWM) 100 in accordance with the present technology. The electronic circuit required to provide operational enablement of the PWM 100 may, in an exemplary embodiment, correspond to a microprocessor 310, associated sensors 320-326, display device 340 and other elements as will be described later. Microprocessor 310 may correspond to a microcontroller depending on the desired operational capabilities of the PWM 100. For example, if a relatively simple construction or model of PWM 100 is desired, for example, one capable of limited operation, a microcontroller may be used. A more functionally advanced model of PWM 100 may require more sophisticated onboard processing capability and, thus, may require the use of a microprocessor.

Operation of the PWM 100 may be characterized as corresponding to a series of measurement sweeps made in periodic intervals. In an exemplary configuration, such measurement sweeps may be performed every 20 seconds, i.e., 3 times per minute. Measurements made during each sweep are stored in non-volatile memory 312 that may correspond to a portion of microprocessor 310 or may be provided as a separate internal memory located within the PWD 100. In either case the memory is sized to provide adequate data storage for a preselected time frame based on the number and types of measurements to be taken. In an exemplary embodiment of the PWM 100 the memory is selected to provide sufficient storage to store at least 7 days of activity and providing for at least 8 bytes of information per measurement sweep and may correspond to a memory capacity of at least 254Kbytes.

With further reference to FIG. 3, PWM 100 operational circuitry 300 may include a number of sensors 320-326, each configured to be responsive to selected physiological or biometric properties such as those previously mentioned including heart pulse rate, blood pressure (systolic and diastolic), changes in motion (accelerometer), and other such physiological and biometric measurements as may be deemed important. For example, sensor 320 may correspond to an infrared sensitive diode that may be used to detect blood flow from which may be calculated both heart pulse rate and blood pressure.

Sensor 322 may correspond to a multi-axis accelerometer whose outputs may be used to calculate relative motion of the user. In some embodiments of the present technology such motion calculations may also be use to automatically determine the type of activity the user is undertaking. Such automatic activity detection may be employed in an “auto select” mode as previously mentioned or, in some embodiments of the present technology, may be provided as the sole method of operational mode selection.

Sensor 324 may correspond to a temperature sensor for detection the body temperature of a user, while sensor 326 may correspond to a moisture sensitive sensor that may provide information relative to body moisture loss. Alternative choices of sensors as well as additional (or fewer) sensors may be selected as desired or necessary to provide data input to the PWM 100 as necessary for its particular design.

Measurements are correlated to both the sweep (i.e.; measurements made each 20 seconds) and modes (i.e.; “Walking”) in which they occur and may be stored as individual sequential records in non-volatile memory 312. If sufficient storage is available within non-volatile memory 312, a relational data structure may be established to provide easier correlation of data relative to the individual user.

Each mode has associated with it an optimal pulse rate (OPR) for the user, indicating the state of the body achieved during the activity. This OPR is based on factors maintained in the PWM 100 such as age, sex, height, Body Mass Index (BMI), Body Impedance Analysis (BIA) fat content, athletic condition, and such other wellness and physiological measurement requirements as deemed necessary. Entry of these data may be accomplished manually by operation of mode switch 350 (corresponding to mode switch 130 of FIG. 1) and control switches 352, 354 illustrated in FIG. 3 or downloaded to the PWM 100 by a data station as will be explained more fully later with respect to FIG. 4.

In order to calibrate PWD 100, the user's resting pulse rate (RPR) is obtained for a short period following rest and stored in non-volatile memory 312. It is assumed that the resting pulse rate is a valid indication of the user's physiological resting state and can be correlated to the user's basal metabolic rate (BMR). Other physiological parameters may be used in addition to RPR to calculate BMR as deemed appropriate. For continued accuracy of measurement, recalibration of RPR may be performed at least once a week. The RPR may be used as a baseline for data analysis and historical comparisons.

In order to correlate the user's optimal pulse rate (OPR) to a particular mode, the PWM 100 may utilizes a series of light-emitting diodes 142 (FIG. 1) or other visual cues to provide feedback as the user moves towards (and eventually achieves) OPR. Visual cues may include monitor-based histograms and other such cues as are deemed appropriate or necessary, including audio and tactile feedback cues. For example, if a user has an OPR of 85 for the mode of “walking,” the PWM may indicate a 50% OPR as measured against the RPR by illuminating an amber LCD, and 100% of OPR as measured against RPR by illuminating a green LCD. The green LCD would continue to be illuminated for as long as the user maintained OPR for the Mode.

Certain modes necessitate going from a higher pulse rate to the resting pulse rate (RPR), which then becomes the optimal pulse rate (OPR). For example, if a user has an OPR (RPR) of 48 for the mode of “meditating”, and the user enters the mode at a pulse rate of 80, the PWM may indicate a 50% OPR by illuminating an amber LCD, and 100% of OPR by illuminating a green LCD. The green LCD would continue to be illuminated for as long as the user maintained OPR (RPR) for the mode.

PWM 100 may display a “Wellness Score” that may be calculated based, for example, on a correlation of body mass index (BMI) with selected “wellness” changes exhibited by the User over time. The score may be based on a logarithmic scale of, for example, 0 to 1000, with exponential improvement being shown at as BMI approaches optimal values for the user. The score may be calculated for the individual user based on the percentage of time the OPR is maintained during the course of the day and the mode being measured. This Wellness Score is available to the user at any time on the display panel 140 (FIG. 1) by selecting the “score” mode using mode switch 130.

With further reference to FIG. 3, PWM 100 contains a Radio-frequency Identification (“RFID”) chip 360 or other non-invasive and contact less user identification system that uniquely identifies the user. PWM 100 may also include a contact less smart chip 370 (CSC) or other central processing unit with associated memory and integrated bus that enables the PWM 100 to perform bi-directional data communication with properly equipped external devices and systems, for example, Data Stations. Finally, PWM 100 may be battery operated by way of battery 380. Battery 380 may be a rechargeable batter and may be recharge as a function of the data exchange process through Data Stations or may be recharged by way of a separate battery charger device (not shown) including a built-in solar cell charging arrangement mounted on PWM 100.

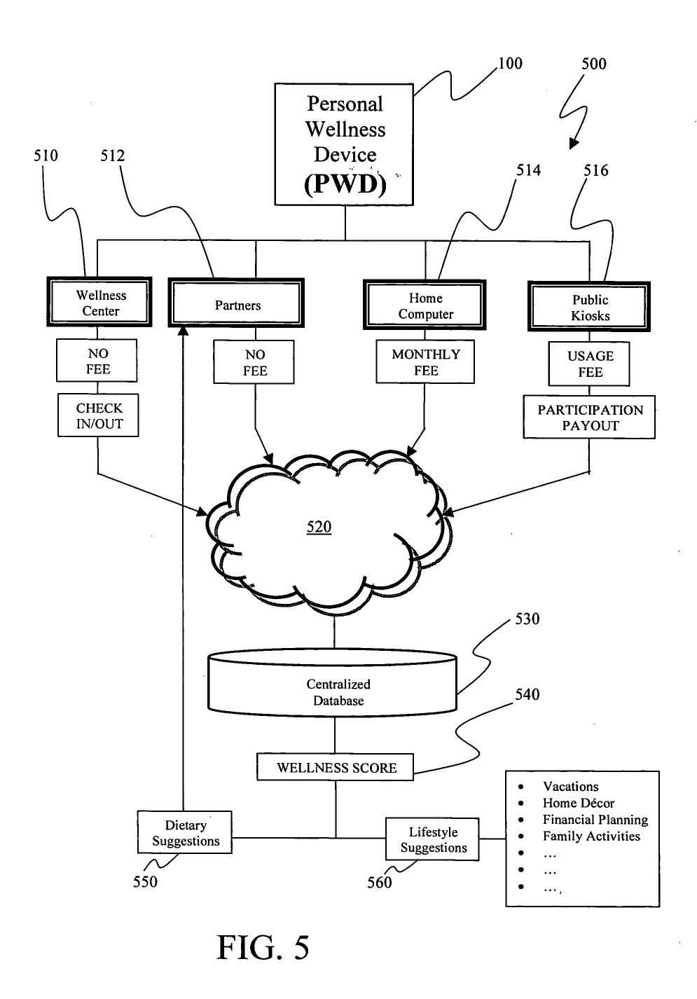

Data Stations may correspond to a stand-alone kiosk 400 as illustrated in FIG. 4 or computer connectivity ports or devices such as a USB (or other data transfer type) computer connection that allow the exchange of user data from the PWM 100 to the Data Station. When user data has been exchanged from the PWM 100, the non-volatile memory 312 may be flushed and data acquisition in the PWM 100 begun again. With reference to FIG. 4, stand-alone kiosks 400 may be provided at various locations including both public and private locations. For example, in accordance with some embodiments of the present technology, stand-alone kiosks 400 may be placed in shopping malls or other public locations to afford users the opportunity to download data from their PWM 100 as well as to receive data from a central database as will be described later with respect to FIG. 5.

Stand-alone kiosk 400 may correspond to a platform 410 supporting a display panel 420 by way of support columns 412, 414. Display panel 420 may include a display device 430 as well as a number of control devices and/or communication ports 440-448 used to activate the stand-alone kiosk 400 and provide communications between a PWM 100 and the stand-alone kiosk 400. Communication between PWM 100 and the stand-alone kiosk may be carried out by direction connection of PWM 100 to a port on the kiosk, or by other, non-contact type connections including, but not limited to, WiFi, Bluetooth, optical, or inductive coupling. The stand-alone kiosk 400 may be coupled to the central database via a telephone line and modem or by some other available communication connection including, but not limited to, direct, dedicated lines, ethernet connections, WiFi and Internet connections.

With reference now to FIG. 5, there is diagrammatically illustrated an operational overview and system flow diagram 500 of the general operation of the data gathering and evaluation aspects of the present technology. As illustrated, various Data Stations 510, 512, 514, 516 may be configured to relay PWM 100 data to a central database 530 (Central DB). The various Data Stations may be associated with a number of different facets of the general operation of the data gathering and evaluation aspects of the present technology. In an exemplary embodiment of the present technology, one or more of Data Stations 510, 512, 514, 516 may be provided. It should be understood that although only a single one of each of the illustrated types of Data Stations is illustrated, multiple ones of such Data stations might be provided.

In the exemplary embodiment of the present technology illustrated in FIG. 5, Data Station 516 may correspond to previously mentioned stand-alone kiosks 400 that may be placed in a public location such as a shopping mall or other public area. Data Station 510 and 512 may be physically similarly configured to stand-alone kiosks 400 but may be place in alternate, private locations corresponding to a Wellness Center 510 or other Partner location 512. Data Station 514 may be configured to correspond to a home use data transfer arrangement to be associated with a home computer.

As may be seen from operational overview and system flow diagram 500, certain operational differences may be applied depending on Data Station location. Data Stations located at Wellness Center 510 and Partner location 512 may be configured so as not to collect a fee from users for use of the Data Station as such use may be provided through membership agreements between the user and owners of the location. Data Stations at Public Kiosks 516 may be configured to collect a usage fee while a Data Station associated with Home Computer 514 may be configured to transfer data based on a monthly fee arrangement with the Central DB provider.

Regardless of the Data Station used, data from PWM 100 may be transferred to Central DB 530 via a communication link 520 that may correspond to any of the previously mentioned communications methodologies including telephone modem, direct, and Internet connection or other communications methodologies as may be available or made available. Upon receipt of data from PWM 100 at the Central DB 530, the data may be converted to sequential User records and stored in a relational database format (RDBMS) where at least a User ID, Mode, and Date/Time of Measurement may be used as primary keys along with the User's unique ID (as maintained in the RFID chip 360).

The Central DB 530 correlates the time-based measurements with mode to create time-based activity and physiology charts, i.e., Wellness Charts. These Wellness Charts may be made available to the user, and can be viewed in a number of ways including, but not limited to, optimal pulse rate (OPR) by Mode, OPR by time of day, OPR by day of week, and/or OPR by month. Review of the User's Wellness Charts 540 may provide insight into subtle changes in the User's physiology, such as slight irregular variations in the resting pulse rate (RPR) and a powerful window into the User's physiology over time. In addition, evaluation of the User's Wellness Charts 540 may be used to provide Dietary Suggestions 550 or Lifestyle Suggestions 560 to PWM users through selected Data Stations, e.g., Data Station 512 associated with a Partners location. Lifestyle Suggestions 560 may involve a variety of suggestion areas that may be related to or associated with possible areas in which improvements or adjustments in the user's lifestyle may be made in order to advance a user's goal of improved personal wellness.

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

1. A personal wellness monitor, comprising:
a housing,
a human body appendage wearable support for said housing;
at least one biometric sensor mounted in said housing and configured to produce biometric data;
a memory mounted in said housing;
a microprocessor mounted in said housing and configured to receive said biometric data from said biometric sensor and to cause said biometric data to be stored in said memory; and
a means for transmitting data stored in said memory to a remote database.

2. The personal wellness monitor of claim 1, further comprising:
a display mounted on said housing,
wherein said display is configured to provide visual indications related to the biometric data produced by said biometric sensor.

3. The personal wellness monitor of claim 2, further comprising:
means for receiving data from a remote database,
wherein said display is further configured to provide a visual indication of data received from a remote database.

4. The personal wellness monitor of claim 1, wherein said microprocessor is configured to cause said biometric data to be stored in different portions of said memory according to one or more different types of human activity.

6. The personal wellness monitor of claim 4, further comprising:
a mode selection means whereby the one or more different types of human activity may be designated.

7. The personal wellness monitor of claim 6, wherein said mode selection means comprises a mode selection switch mounted on said housing.

8. The personal wellness monitor of claim 6, wherein said mode selection means comprises a mode selection signal generated in response to said biometric data.

9. A personal wellness monitoring system, comprising:
a human body appendage wearable support device;
a housing supported by said support device;
a biometric data producing sensor mounted within said housing;
a memory housed within said housing and configured to receive biometric data from said biometric data producing sensor;
a display mounted in said housing and configured to provide a visual display of data;
a remote database configured to receive and evaluate biometric data; and
a data transmitter and receiver mounted in said housing and configured to transmit data to and receive data from said remote database,
whereby biometric data may be transmitted to said remote database for evaluation and data based upon the evaluation may be transmitted to said display.

10. The personal wellness monitoring system of claim 9, further comprising:
a data station, said data station configured to receive data from said data transmitter and to transmit data to said remote database and further configured to receive data from said remote database and transmit data to said receiver.

11. The personal wellness monitoring system of claim 10, wherein said data station is configured to be operated by a user and to receive a fee from said user prior to transmitting data to said remote database.

12. The personal wellness monitoring system of claim 9, wherein said biometric data producing sensor comprises an infrared sensitive sensor.

13. The personal wellness monitoring system of claim 9, wherein the data transmitted to said display comprises dietary information.

14. The personal wellness monitoring system of claim 9, wherein the data transmitted to said display comprises lifestyle information.

15. A method of monitoring personal wellness, comprising the steps of:
providing a portable, wearable, battery operated biometric data measuring device;
periodically storing measured biometric data in a selected portion of a memory device based on designated human activities;
transmitting stored data to a remote database;
evaluating the transmitted data at said remote database; and
transmitting data based upon said evaluation to said device.

16. The method of claim 15, further comprising the steps of:
designating the type of human activity being undertaken; and
storing measured biometric data in selected separate portions of a memory device based on selected different designated human activities.

17. The method of claim 16, wherein said step of designating is performed manually.

18. The method of claim 16, wherein said step of designating is performed automatically based on the results of analysis of the measured biometric data.
